(19) United States
(12) Patent Application Publication
(10) Pub. No.: US 2025/0367114 A1
(43) Pub. Date: Dec. 4, 2025

(54) INHALABLE COMPOSITION FOR ENHANCING ALERTNESS AND VITALITY AND DEVICE FOR SUPPLYING THE INHALABLE COMPOSITION

(71) Applicant: KT&G CORPORATION, Daejeon (KR)

(72) Inventors: Young Chul LEE, Daejeon (KR); So Eun KIM, Daejeon (KR); Sung Ok MOON, Daejeon (KR); Bo Young LEE, Daejeon (KR); Hwa Chul JUNG, Daejeon (KR); Han Jae SHIN, Daejeon (KR)

(73) Assignee: KT&G CORPORATION, Daejeon (KR)

(21) Appl. No.: 19/193,021

(22) Filed: Apr. 29, 2025

(30) Foreign Application Priority Data
May 31, 2024 (KR) 10-2024-0071780

(51) Int. Cl. A61K 9/00; A61K 31/455; A61K 31/52; A61K 31/7004; A61K 33/06; A61P 25/26

(52) U.S. Cl. CPC A61K 9/0075; A61K 9/0078; A61K 9/008; A61K 31/455; A61K 31/52; A61K 31/7004; A61K 33/06; A61P 25/26

(57) ABSTRACT

In an inhalable composition for enhancing alertness and vitality according to an embodiment, the composition includes caffeine and an enhancing agent including one or more selected from glucose and magnesium.

### CROSS-REFERENCE TO RELATED APPLICATION

This application is based on and claims priority under 35 U.S.C. § 119 to Korean Patent Application No. 10-2024-0071780, filed on May 31, 2024, in the Korean Intellectual Property Office, the disclosure of which is incorporated by reference herein in its entirety.

### BACKGROUND

### 1. Field

The disclosure relates to an inhalable composition for enhancing alertness and vitality and including a relatively low content of caffeine and a device for supplying the inhalable composition.

### 2. Description of the Related Art

Caffeine is widely used as a substance to enhance alertness and vitality by acting on the central nervous system, but it irritates the esophagus by relaxing the lower esophageal sphincter and promoting gastric acid secretion. Accordingly, the development of an inhaling agent that is administered through the respiratory tract instead of oral administration has been conducted.

However, a method of inhaling caffeine through the respiratory tract may affect the human body at a much faster speed ratio than ingesting caffeine through food, etc. and excessive intake thereof may cause side effects such as headache, dizziness, sleep disorder, palpitation, and muscle cramps.

### SUMMARY

Provided are an inhalable composition for enhancing alertness and vitality and a device for supplying the inhalable composition.

Embodiments may enhance an alertness and vitality effect while lowering a content of caffeine.

Objects to be solved by the embodiments are not limited to the above objects, and objects not mentioned may be understood by those skilled in the art to which the embodiments belong from the specification and the attached drawings.

Additional aspects will be set forth in part in the description which follows and, in part, will be apparent from the description, or may be learned by practice of the presented embodiments of the disclosure.

According to an embodiment, a inhalable composition configured to enhance alertness and vitality may include caffeine and an enhancing agent including one or more selected from glucose and magnesium.

According to another embodiment, a device for supplying an inhalable composition may include a storage container storing the inhalable composition described above and an inhaler coupled to the storage container.

### BRIEF DESCRIPTION OF THE DRAWINGS

The above and other aspects, features, and advantages of certain embodiments of the disclosure will be more apparent from the following description taken in conjunction with the accompanying drawings, in which:

FIGS. 1A-1D are graphs showing results of general behavioral experiments with respect to inhalable compositions of comparative examples and embodiments.

### DETAILED DESCRIPTION

Regarding the terms in the various embodiments, the general terms which are currently and widely used are selected in consideration of functions of structural elements in the various embodiments of the present disclosure. However, meanings of the terms can be changed according to intention, a judicial precedence, the appearance of a new technology, and the like. In addition, in certain cases, terms which can be arbitrarily selected by the applicant in particular cases. In such a case, the meaning of the terms will be described in detail at the corresponding portion in the description of the present disclosure. Therefore, the terms used in the various embodiments of the present disclosure should be defined based on the meanings of the terms and the descriptions provided herein.

In addition, unless explicitly described to the contrary, the word “comprise” and variations such as “comprises” or “comprising” will be understood to imply the inclusion of stated elements but not the exclusion of any other elements. In addition, the terms “-er”, “-or”, and “module” described in the specification mean units for processing at least one function and operation and can be implemented by hardware components or software components and combinations thereof.

As used herein, hen an expression such as “at least any one” precedes arranged elements, it modifies all elements rather than each arranged element. For example, the expression “at least any one of a, b, and c” should be construed to include a, b, c, or a and b, a and c, b and c, or a, b, and c.

Hereinafter, the present disclosure will now be described more fully with reference to the accompanying drawings, in which exemplary embodiments of the present disclosure are shown such that one of ordinary skill in the art may easily work the present disclosure. But, the present disclosure may be implemented in a form that can be implemented in various different forms, and is not limited to the embodiments described herein.

Throughout the specification, embodiments are provided as arbitrary distinctions to facilitate explanation of the disclosure, and each embodiment is not necessarily exclusive of the others. For example, configurations disclosed in an embodiment may be applied and/or implemented in other embodiments, and may be applied and/or implemented with modifications without departing from the scope of the disclosure.

Also, terms used in the disclosure are only for the purpose of describing embodiments and are not intended to limit the embodiments. In the disclosure, singular forms also include plural forms, unless specifically stated otherwise.

According to an embodiment, a inhalable composition configured to enhance alertness and vitality may be provided.

A inhalable composition configured to enhance alertness and vitality according to an embodiment may include caffeine and an enhancing agent including one or more selected from glucose and magnesium to provide an alertness and vitality enhancement effect with a relatively low content of caffeine.

For example, based on the total weight of the inhalable composition, caffeine may be included in a content of 15 wt % or less. Alternatively, based on the total weight of the inhalable composition, caffeine may be included in a content of 8 wt % or less. When caffeine exceeds the above content, the content of caffeine may be too high to cause problems of side effects, such as headache, dizziness, sleep disorder, palpitation, and muscle cramps.

In an embodiment, the enhancing agent may be one or more selected from glucose and magnesium. The enhancing agent may improve an alertness and vitality enhancement effect by being included in the inhalable composition together with caffeine.

The enhancing agent may include glucose or magnesium, or may include both glucose and magnesium. When both glucose and magnesium are included as the enhancing agent, the alertness and vitality enhancement effect of the inhalable composition according to an embodiment may be further improved.

The inhalable composition configured to enhance alertness and vitality according to an embodiment may further include vitamin B3 and a solvent, in addition to the caffeine and the enhancing agent described above.

For example, the caffeine and the vitamin B3 may be included in a weight ratio of 2:1 to 2:3. When the content of vitamin B3 is less than the above range, the caffeine may not be sufficiently dissolved in the inhalable composition. When the content of vitamin B3 is greater than the above range, an effect of increasing the solubility of caffeine may be minimal.

The inhalable composition configured to enhance alertness and vitality according to an embodiment may include, based on the total weight of the composition, 2 wt % to 15 wt % of the caffeine, 1 wt % to 20 wt % of the enhancing agent, and 45 wt % to 96 wt % of the solvent.

In an embodiment, the solvent may dissolve the caffeine, the vitamin B3, and the enhancing agent, and may be included in an amount equal to the remaining amount of the total weight of the composition, excluding the caffeine, the vitamin B3, and the enhancing agent. In an embodiment, the solvent may be included in a content of 45 wt % to 96 wt %, based on the total weight of the inhalable composition. In an embodiment, the solvent may be included in a content of 55 wt % to 85 wt %, based on the total weight of the inhalable composition. In an embodiment, the solvent may be included in a content of 70 wt % to 80 wt %, based on the total weight of the inhalable composition.

The solvent included in the inhalable composition according to an embodiment may include any one or more water, alcohol, propylene glycol, and vegetable glycerin. The solvent may dissolve the caffeine, the vitamin B3, and the enhancing agent.

The inhalable composition according to an embodiment may further include sodium chloride in the solvent. For example, the solvent may include water and sodium chloride. The sodium chloride may be included in a content of 0.5 wt % to 1.5 wt %, the total weight of water. Also, the sodium chloride may be included in a content of 0.85 wt % to 0.95 wt %, the total weight of the water.

For example, the solvent included in the inhalable composition may include 98.5 wt % to 99.5 wt % of water and 0.5 wt % and 1.5 wt % of sodium chloride, based on the total weight of the solvent.

In the inhalable composition according to an embodiment, the enhancing agent may include 1 wt % to 20 wt % of glucose, based on the total weight of the composition. Alternatively, the enhancing agent may include 1 wt % to 20 wt % of magnesium, based on the total weight of the composition.

Also, the enhancing agent may include 1 wt % to 20 wt % of glucose and magnesium, based on the total weight of the composition. When the enhancing agent includes both glucose and magnesium, the enhancing agent may include 1 wt % to 10 wt % of glucose and 1 wt % to 10 wt % of magnesium. Also, the enhancing agent may include 1 wt % to 8 wt % of glucose and 1 wt % to 8 wt % of magnesium, or may include 1 wt % to 5 wt % of glucose and 1 wt % to 5 wt % of magnesium.

The inhalable composition according to an embodiment may further include an additive usable in the inhalable composition. For example, the inhalable composition may further include one or more flavoring agents or flavoring ingredients. As another example, the inhalable composition may further include a propellant when the inhalable composition is used in a metered-dose inhaler. However, the disclosure is not limited thereto.

According to an embodiment, a device for supplying a inhalable composition, which includes a storage container in which the inhalable composition described above is accommodated and an inhaler coupled to the storage container. For example, the storage container may be removably coupled to the inhaler. As another example, the storage container may be fixed to the inhaler and include an opening or the like capable of filling the inhalable composition.

The inhalable composition configured to enhance alertness and vitality according to an embodiment may be in a form that may be inhaled through an inhaler. The inhaler may have various forms, such as a metered-dose inhaler, a dry powder inhaler, a nebulizer, or the like.

The inhalable composition may be in a form of a solid, a liquid, or a mixture of a solid and a liquid. For example, the inhalable composition may be in a form of a mixture of solid powders of the caffeine and the enhancing agent. Alternatively, the inhalable composition may be in a form of a solid obtained by dissolving the caffeine, the vitamin B3, and the enhancing agent in the solvent and then drying the solvent. The composition in the solid form may be manufactured as a dry powder that may be inhaled through a dry powder inhaler.

As another example, the inhalable composition may be in a form of a liquid obtained by dissolving the caffeine, the vitamin B3, and the enhancing agent into the solvent. According to an embodiment, the inhaler may be a nebulizer. The nebulizer may atomize the inhalable composition in the liquid form, and the atomized composition may be administered to the respiratory tract of a human. According to an embodiment, an alertness and vitality enhancement effect may be obtained by inhaling the atomized inhalable composition.

According to an embodiment, the inhaler may include an atomizer atomizing the inhalable composition, a battery configured to supply power to the atomizer, and a supply port providing the atomized composition to the atomizer. For example, the inhaler including the atomizer, the battery, and the supply port as described above may be a nebulizer.

For example, the atomizer may be a jet type that uses compressed air to atomize a liquid composition, an ultrasonic type that uses high-frequency sound waves to atomize a liquid composition, a vibrating type that applies vibration to a mesh to pass a liquid composition through the mesh and make the liquid composition into fine particles, or a heating type that atomizes a liquid composition through heating.

Also, the inhaler according to an embodiment may adjust an atomization rate by including a heating element and operating the heating element. The heating element may be controlled by a separate switch provided outside the device for supplying an inhalable composition, but is not limited thereto.

According to an embodiment, the inhalable composition atomized by the atomizer may be administered to the respiratory tract of a human through the supply port. The supply port may include a mouthpiece or mask arranged adjacent to the supply port. The mouthpiece or mask may be physically connected to the supply port.

For example, the supply port may be connected to a separate mouthpiece, or a portion integrally extending from the supply port may be a mouthpiece. As another example, the supply port may include a mask for inhalation connected to the supply port. The mask for inhalation may have a shape that is in close contact with the face.

Hereinafter, a more detailed description is given with reference to embodiments and experimental examples. These embodiments are intended to be illustrative only, and it will be apparent to those skilled in the art that the disclosure is not to be construed as being limited by the embodiments.

### EMBODIMENTS

An inhalable composition was manufactured and an alertness and vitality enhancement effect thereof was measured. In detail, the inhalable composition was manufactured and administered orally or by inhalation to experimental animals, and then the alertness and vitality enhancement effect was measured through general behavioral experiments and forced swimming ability experiments.

### 1. Preparation for Tests

### (1) Preparation for Experimental Animals

The experimental animals were C57BL/6 mice (male, 10 weeks old) purchased from KOATECH after receiving approval from the Institutional Animal Care and Use Committee (IACUC). The experimental animals were acclimatized for a week in the breeding room of the bio-efficacy evaluation center of KT&G (temperature 23±2° C., humidity 50±10%, light-dark cycle 12 hours) and then used in the experiments.

### (2) Verification of an Effect of Caffeine Through Oral Administration

Caffeine was dissolved in a 0.9% saline solution and administered orally to the mice by using Zonde at a dosage of 10 ml/kg, and then general behavioral experiments were conducted. Samples of experimental examples 1 to 3 below were orally administered to the C57BL/6 mice, and then the number of movements, the speed of movement (mm/s), the distance of movement (meter), and the time of movement (sec) for an hour were measured using a LABORAS behavioral device.

Experimental example 1 was a control group, in which only 10 ml/kg of a 0.9% saline solution was orally administered. In experimental example 2, caffeine was dissolved in a 0.9% saline solution at a dose of 5 mg/kg and orally administered by using Zonde. In experimental example 3, caffeine was dissolved in a 0.9% saline solution at a dose of 30 mg/kg and orally administered by using Zonde. With respect to each of experimental examples 1 to 3, the alertness effect of caffeine was verified through general behavioral experiments, and results thereof are shown in Table 1 below.

TABLE 1

| | Average of general behavioral experiments | | | | | | | |
|---|---|---|---|---|---|---|---|---|
| | Number | | | | Significance of behavioral | | | |
| | of | | | | experiments | | | |
| | times | Speed | Distance | | Number | | | |
| Sample | (count) | (mm/s) | (meter) | Time (sec) | of times | Speed | Distance | Time |
| Experimental | 485.25 ± 158.68 | 4.21 ± 1.81 | 15.16 ± 6.5 | 389.2 ± 153.71 | | | | |
| example | | | | | | | | |
| 1 | | | | | | | | |
| Experimental | 532.5 ± 66.33 | 5.689 ± 0.91 | 20.48 ± 3.27 | 405.65 ± 21.89 | 0.6 | 0.19 | 0.19 | 0.84 |
| example | | | | | | | | |
| 2 | | | | | | | | |
| Experimental | 1034.5 ± 220.05 | 9.33 ± 2.18 | 33.59 ± 7.85 | 884.15 ± 154.86 | 0.007 | 0.01 | 0.01 | 0.004 |
| example | | | | | | | | |
| 3 | | | | | | | | |

As the results of measurement, it was confirmed that there was almost no effect in experimental example 2 in which caffeine was orally administered at a concentration of 5 mg/kg, and an alertness effect was observed in experimental example 3 in which caffeine was orally administered at a concentration of 30 mg/kg.

### 2. General Behavioral Experiments for Compositions for Inhalation

Compositions for inhalation of comparative examples and embodiments were manufactured, and were administered to the mice through inhalation by atomizing the compositions with a mist generator. The inhalation time (min) at which the efficacy was observed was measured according to the composition, and the composition was collected for 10 minutes by using a sampling pump and Cambridge filer and a total particulate matter (TPM) concentration thereof was calculated by gravimetric measurement.

The compositions for inhalation of comparative examples 1 to 5 and embodiments 1 to 3 in Table 2 below were administered to the C57BL/6 mice by inhalation, and then the number of movements, the speed of movement (mm/s), the distance of movement (meter), and the time of movement (sec) for an hour were measured using a LABORAS behavioral device. All materials included in the compositions for inhalation of comparative examples and embodiments were dissolved in a 0.9% saline solution and used.

In a control group with respect to each comparative example and embodiment, a 0.9% saline solution was atomized by a mist generator and administered to the mice by inhalation.

TABLE 2

| | | | | | | | | Significance of | | | |
|---|---|---|---|---|---|---|---|---|---|---|---|
| | | | | Average of general behavioral experiments | | | | behavioral experiments | | | |
| | | Inhalation | Con- | Number of | | | | Num- | | | |
| | Com- | time | centration | times | Speed | Distance | | ber of | | | |
| Sample | position | (min) | (mg/L) | (count) | (mm/s) | (meter) | Time (sec) | times | Speed | Distance | Time |
| Experi- | Control | 50 | | 498.25 ± 50.37 | 6.21 ± 0.93 | 22.35 ± 3.34 | 462.86 ± 55.4 | | | | |
| mental | group | | | | | | | | | | |
| exam- | Com- | 50 | 0.054 | 638.25 ± 169.01 | 7.79 ± 1.37 | 28.04 ± 4.94 | 588.02 ± 118.71 | 0.16 | 0.105 | 0.105 | 0.104 |
| ple | para- | | | | | | | | | | |
| 4 | tive | | | | | | | | | | |
| | exam- | | | | | | | | | | |
| | ple 1 | | | | | | | | | | |
| Experi- | Control | 40 | | 840.25 ± 136.04 | 9.56 ± 1.52 | 34.41 ± 5.47 | 758.09 ± 90.4 | | | | |
| mental | group | | | | | | | | | | |
| exam- | Com- | 40 | 0.436 | 960.75 ± 274.31 | 11.3 ± 3.19 | 40.69 ± 11.48 | 839.85 ± 233.26 | 0.461 | 0.362 | 0.362 | 0.537 |
| ple | para- | | | | | | | | | | |
| 5 | tive | | | | | | | | | | |
| | exam- | | | | | | | | | | |
| | ple 2 | | | | | | | | | | |
| Experi- | Control | 60 | | 673.33 ± 41.06 | 7.75 ± 1.8 | 26.9 ± 5.67 | 607.11 ± 63.47 | | | | |
| mental | group | | | | | | | | | | |
| exam- | Com- | 30 | 0.22 | 858.25 ± 151.56 | 10.56 ± 1.93 | 37.09 ± 6.2 | 752.05 ± 110.17 | 0.1 | 0.1 | 0.051 | 0.1 |
| ple | para- | | | | | | | | | | |
| 6 | tive | | | | | | | | | | |
| | exam- | | | | | | | | | | |
| | ple 3 | | | | | | | | | | |
| | Com- | 60 | 0.23 | 744 ± 155.8 | 9.22 ± 2.49 | 33.19 ± 8.95 | 663.22 ± 111.05 | 0.487 | 0.429 | 0.28 | 0.473 |
| | para- | | | | | | | | | | |
| | tive | | | | | | | | | | |
| | exam- | | | | | | | | | | |
| | ple 3 | | | | | | | | | | |
| Experi- | Control | 40 | | 676.25 ± 110.18 | 8.61 ± 1.84 | 32.97 ± 9.36 | 621.82 ± 86.51 | | | | |
| mental | group | | | | | | | | | | |
| exam- | Com- | 20 | 0.52 | 820.5 ± 121.35 | 9.06 ± 0.88 | 30.98 ± 6.64 | 688.79 ± 60.66 | 0.128 | 0.671 | 0.741 | 0.25 |
| ple | para- | | | | | | | | | | |
| 7 | tive | | | | | | | | | | |
| | exam- | | | | | | | | | | |
| | ple 4 | | | | | | | | | | |
| | Com- | 40 | 0.54 | 823 ± 158.63 | 9.07 ± 1.68 | 53.04 ± 8.31 | 702.98 ± 126.99 | 0.179 | 0.722 | 0.018 | 0.33 |
| | para- | | | | | | | | | | |
| | tive | | | | | | | | | | |
| | exam- | | | | | | | | | | |
| | ple 4 | | | | | | | | | | |
| Experi- | Control | 30 | | 828.25 ± 337.72 | 12.16 ± 2.27 | 43.76 ± 8.18 | 803.48 ± 77.35 | | | | |
| mental | group | | | | | | | | | | |
| exam- | Com- | 10 | 0.56 | 950.25 ± 99.26 | 12.8 ± 4.97 | 43.83 ± 5.65 | 852 ± 108.4 | 0.514 | 0.821 | 0.989 | 0.493 |
| ple | para- | | | | | | | | | | |
| 8 | tive | | | | | | | | | | |
| | exam- | | | | | | | | | | |
| | ple 5 | | | | | | | | | | |
| | Com- | 20 | 0.594 | 1121.75 ± 326.81 | 13.35 ± 5.79 | 48.08 ± 20.84 | 1067.82 ± 326.19 | 0.258 | 0.713 | 0.713 | 0.166 |
| | para- | | | | | | | | | | |
| | tive | | | | | | | | | | |
| | exam- | | | | | | | | | | |
| | ple 5 | | | | | | | | | | |
| | Com- | 30 | 0.558 | 1390.25 ± 198.39 | 17.92 ± 1.95 | 64.5 ± 7.02 | 1279.34 ± 152.84 | 0.03 | 0.008 | 0.008 | 0.001 |
| | para- | | | | | | | | | | |
| | tive | | | | | | | | | | |
| | exam- | | | | | | | | | | |
| | ple 5 | | | | | | | | | | |
| Experi- | Control | 15 | | 771.75 ± 169.16 | 9.22 ± 1.64 | 33.2 ± 5.89 | 656.39 ± 70.82 | | | | |
| mental | group | | | | | | | | | | |
| exam- | Embodi- | 5 | 1.01 | 833.25 ± 268.13 | 9.4 ± 2.799 | 33.84 ± 10.03 | 672.89 ± 184.35 | 0.711 | 0.92 | 0.92 | 0.872 |
| ple | ment 1 | | | | | | | | | | |
| 9 | Embodi- | 10 | 1.01 | 1164.5 ± 97.12 | 13.53 ± 1.63 | 48.69 ± 5.86 | 977.13 ± 111.41 | 0.007 | 0.01 | 0.009 | 0.03 |
| | ment 1 | | | | | | | | | | |
| | Embodi- | 15 | 1.01 | 1296 ± 135.33 | 13.84 ± 1.28 | 49.83 ± 4.59 | 1087.33 ± 113.13 | 0.03 | 0.008 | 0.008 | 0.001 |
| | ment 1 | | | | | | | | | | |
| Experi- | Control | 30 | | 882.75 ± 258.96 | 11.52 ± 4.4 | 41.48 ± 15.84 | 761.1 ± 256.4 | | | | |
| mental | group | | | | | | | | | | |
| exam- | Embodi- | 5 | 0.514 | 1056.75 ± 183.96 | 13.48 ± 2.88 | 48.54 ± 10.35 | 933.99 ± 134.49 | 0.315 | 0.484 | 0.483 | 0.277 |
| ple | ment 2 | | | | | | | | | | |
| 10 | Embodi- | 10 | 0.526 | 1059.25 ± 258.22 | 12.32 ± 2.78 | 44.35 ± 9.99 | 909.27 ± 233.62 | 0.371 | 0.769 | 0.769 | 0.42 |
| | ment 2 | | | | | | | | | | |
| | Embodi- | 20 | 0.52 | 1485.5 ± 268.08 | 17.13 ± 2.43 | 61.68 ± 8.73 | 1333 ± 122.9 | 0.017 | 0.06 | 0.066 | 0.006 |
| | ment 2 | | | | | | | | | | |
| | Embodi- | 30 | 0.54 | 1342.75 ± 48.07 | 15.35 ± 1.49 | 55.74 ± 5.36 | 1181.45 ± 91.78 | 0.012 | 0.15 | 0.14 | 0.02 |
| | ment 2 | | | | | | | | | | |
| Experi- | Control | 15 | | 526.75 ± 267.35 | 6.65 ± 3.19 | 23.94 ± 11.48 | 486.9 ± 254.45 | | | | |
| mental | group | | | | | | | | | | |
| exam- | Embodi- | 5 | 0.718 | 921 ± 139.96 | 10.97 ± 0.77 | 37.96 ± 4.48 | 841.95 ± 65.43 | 0.02 | 0.038 | 0.06\| | 0.035 |
| ple | ment 3 | | | | | | | | | | |
| 11 | Embodi- | 10 | 0.776 | 1122.5 ± 101.4 | 11.94 ± 1.79 | 42.98 ± 6.44 | 968.05 ± 96.98 | 0.006 | 0.027 | 0.027 | 0.012 |
| | ment 3 | | | | | | | | | | |
| | Embodi- | 15 | 0.723 | 1001.5 ± 94.94 | 10.98 ± 0.64 | 38.13 ± 1.28 | 840.87 ± 33.98 | 0.015 | 0.037 | 0.049 | 0.008 |
| | ment 3 | | | | | | | | | | |

### (1) Comparative Example 1

An inhalable composition (comparative example 1) including 2 wt % caffeine was manufactured by dissolving the caffeine in a 0.9% saline solution. The composition of comparative example 1 was administered to the mice by inhalation for 50 minutes by being atomized with a mist generator. As an observation result of general behaviors, it was confirmed that there was no significant difference in effect compared to the control group.

### (2) Comparative Examples 2 to 4

An inhalable composition (comparative example 2) including 10 wt % vitamin B3 was manufactured by dissolving the vitamin B3 in a 0.9% saline solution. Also, an inhalable composition (comparative example 3) including 10 wt % glucose was manufactured by dissolving the glucose in a 0.9% saline solution, and an inhalable composition (comparative example 4) including 10 wt % magnesium was manufactured by dissolving the magnesium in a 0.9% saline solution.

The compositions of comparative examples 2 to 4 were administered to the mice by inhalation for 20 minutes to 60 minutes by being atomized with a mist generator. As observation results of general behaviors, it was confirmed that there were no significant difference in effect compared to the control group.

### (3) Comparative Example 5

An inhalable composition (comparative example 5) including 8 wt % caffeine and 10 wt % vitamin B3 was manufactured by dissolving the caffeine and vitamin B3 in a 0.9% saline solution. The composition of comparative example 5 was administered to the mice by inhalation for 10 minutes to 30 minutes by being atomized with a mist generator. As an observation result of general behaviors, it was confirmed that the effect appeared when the composition was inhaled for 30 minutes or more.

### (4) Embodiment 1

An inhalable composition (embodiment 1) including 8 wt % caffeine, 10 wt % vitamin B3, and 5 wt % glucose was manufactured by dissolving the caffeine, vitamin B3, and glucose in a 0.9% saline solution. The composition of embodiment 1 was administered to the mice by inhalation for 5 minutes to 15 minutes by being atomized with a mist generator. As an observation result of general behaviors, it was confirmed that the effect appeared when the composition was inhaled for 10 minutes or more.

### (5) Embodiment 2

An inhalable composition (embodiment 2) including 8 wt % caffeine, 10 wt % vitamin B3, and 5 wt % magnesium was manufactured by dissolving the caffeine, vitamin B3, and magnesium in a 0.9% saline solution. The composition of embodiment 2 was administered to the mice by inhalation for 5 minutes to 30 minutes by being atomized with a mist generator. As an observation result of general behaviors, it was confirmed that the effect appeared when the composition was inhaled for 20 minutes or more.

### (6) Embodiment 3

An inhalable composition (embodiment 3) including 8 wt % caffeine, 10 wt % vitamin B3, 5 wt % glucose, and 5 wt % magnesium was manufactured by dissolving the caffeine, vitamin B3, glucose, and magnesium in a 0.9% saline solution. The composition of embodiment 3 was administered to the mice by inhalation for 5 minutes to 15 minutes by being atomized with a mist generator. As an observation result of general behaviors, it was confirmed that the effect appeared when the composition was inhaled for 5 minutes or more.

### 3. General Behavioral Experiments and Forced Swimming Experiments for Compositions for Inhalation

The general behavioral experiments and forced swimming experiments were conducted on the compositions for inhalation of the control group, comparative example 5, and embodiments 1 to 3. Each composition was inhaled for 15 minutes by being atomized with a mist generator to compare the efficacy under the same conditions.

The general behavioral experiments were conducted in the same manner as the method of the general behavioral experiments in the section 2, described above. For the forced swimming experiments, a transparent cylinder with a diameter of 8 cm and a height of 25 cm was filled with water at a temperature of 25° C., and then the mice were put in the water to acclimate for 10 minutes. Next, after 24 hours, each composition was administered to the mice by inhalation for 15 minutes, the mice were put in a water tank for 6 minutes, and the immobility time (sec) during the last 4 minutes was measured. Measurement results are shown in Table 3 below.

TABLE 3

| | | | | | | Forced | Significance of behavioral experiments | | | | |
|---|---|---|---|---|---|---|---|---|---|---|---|
| | Con- | Average of general behavioral experiments | | | | swimming | Num- | | | | |
| | cen- | Number | | | | immobility | ber | | | | |
| | tration | of times | Speed | Distance | Time | time | of | | | | Forced |
| Sample | (mg/L) | (count) | (mm/s) | (meter) | (sec) | (sec) | times | Speed | Distance | Time | swimming |
| Control | | 663.5 ± 232.81 | 8.01 ± 2.67 | 28.82 ± 9.61 | 574.32 ± 183.22 | 239.16 ± 2.04 | | | | | |
| group | | | | | | | | | | | |
| Compar- | 0.6 | 874.75 ± 253.12 | 9.24 ± 2.26 | 33.26 ± 8.15 | 728.36 ± 187.48 | 232.25 ± 20.35 | 0.265 | 0.508 | 0.507 | 0.281 | 0.42 |
| ative | | | | | | | | | | | |
| exam- | | | | | | | | | | | |
| ple 5 | | | | | | | | | | | |
| Embodi- | 0.614 | 1090.25 ± 209.56 | 13.79 ± 3.56 | 43.15 ± 4.75 | 975.33 ± 235.32 | 223.12 ± 15.03 | 0.03 | 0.04 | 0.036 | 0.03 | 0.02 |
| ment 1 | | | | | | | | | | | |
| Embodi- | 0.618 | 1065.75 ± 188.63 | 12.75 ± 2.34 | 40.51 ± 3.25 | 894.3 ± 172.11 | 230 ± 6.21 | 0.04 | 0.03 | 0.05 | 0.043 | 0.005 |
| ment 2 | | | | | | | | | | | |
| Embodi- | 0.609 | 1254.5 ± 99.56 | 14.43 ± 1.66 | 47.2 ± 3.1 | 1038.93 ± 170.47 | 175 ± 45.29 | 0.003 | 0.006 | 0.01 | 0.009 | 0.008 |
| ment 3 | | | | | | | | | | | |

As a result, in both the general behavioral experiments and the forced swimming experiments, it was confirmed that compositions (embodiments 1 and 2) including glucose or magnesium showed excellent efficacy compared to comparative example 5, and the composition (embodiment 3) including both glucose and magnesium showed very excellent efficacy.

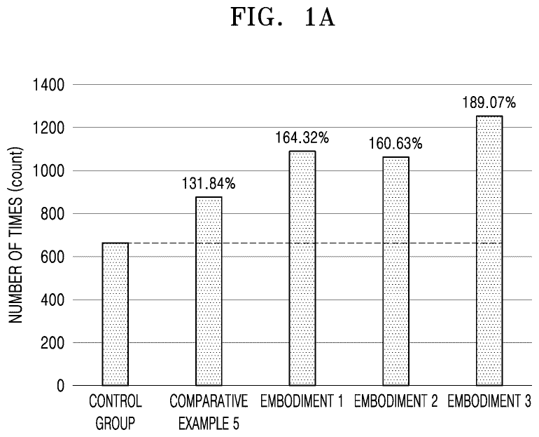

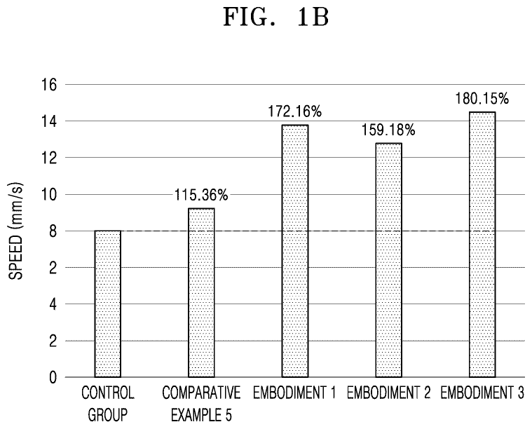

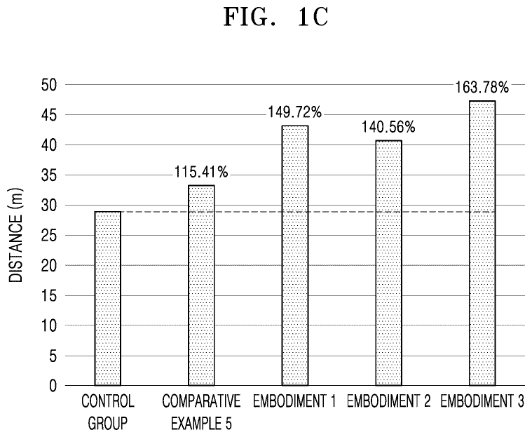

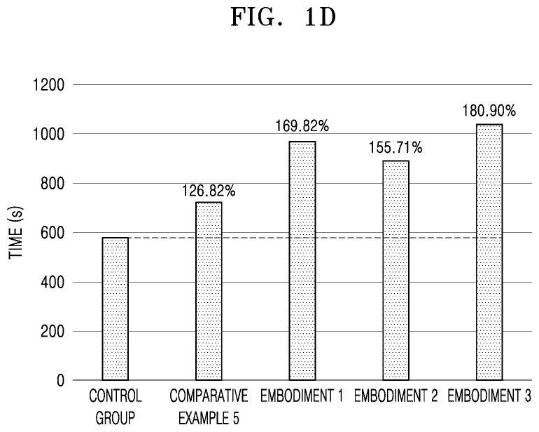

The results of the general behavioral experiments are shown in FIG. 1. FIG. 1A shows the number of movements, FIG. 1B shows the speed of movement, FIG. 1C shows the distance of movement, and FIG. 1D shows the time of movement, in which the measurements were made for an hour by using a LABORAS behavioral device. At the top end of the graph of each of comparative example 5 and embodiments 1 to 3, each increase rate was displayed compared to the control group.

Referring to FIGS. 1A-1D, in the overall general behavioral experimental results, it may be seen that a degree of increase in the effect of Comparative example 5 is minimal compared to the control group, but the increases in the effects of embodiments 1 and 2 are large, and the increase in the effect of embodiment 3 is even greater.

In particular, as can be seen from the results of the forced swimming experiments, the immobility time was slightly shorter in embodiments 1 and 2, and the immobility time was significantly shorter at approximately 175 seconds in embodiment 3, which may confirm an excellent alertness and vitality enhancement effect.

Through the results of the general behavioral experiments and the forced swimming experiments, the alertness and vitality enhancement effects of the compositions for inhalation of embodiments 1 to 3 may be confirmed.

The descriptions of the above-described embodiments are merely examples, and it will be understood by one of ordinary skill in the art that various changes and equivalents thereof may be made. The above-described embodiments may be performed in a different order, and/or the described components may be combined or assembled in a different form, or replaced or substituted with other components or equivalents while still achieving suitable results. Therefore, all differences within the scope equivalent to those described in the claims will be construed as being included in the scope of protection defined by the claims.

A inhalable composition for enhancing alertness and vitality and a device for supplying an inhalable composition including the inhalable composition according to embodiments have an excellent alertness and vitality enhancement effect even at a relatively low content of caffeine, thereby reducing side effects caused by caffeine.

Effects of the present disclosure are not limited to the above effects, and effects that are not mentioned could be clearly understood by one of ordinary skill in the art from the present specification and the attached drawings.

What is claimed is:

1. An inhalable composition for enhancing alertness and vitality, comprising:
caffeine; and
an enhancing agent comprising one or more selected from glucose and magnesium.

2. The inhalable composition of claim 1, wherein, based on a total weight of the inhalable composition, a content of caffeine is 15 wt % or less.

3. The inhalable composition of claim 1, wherein, based on a total weight of the inhalable composition, a content of caffeine is 8 wt % or less.

4. The inhalable composition of claim 1, further comprising:
vitamin B3; and
at least one solvent selected from a group comprising water, alcohol, propylene glycol, and vegetable glycerin.

5. The inhalable composition of claim 4, wherein a weight ratio of the caffeine to the vitamin B3 is 2:1 to 2:3.

6. The inhalable composition of claim 4, wherein, based on a total weight of the inhalable composition,
a content of caffeine is 2 wt % to 15 wt %,
a content of vitamin B3 is 1 wt % to 15 wt %,
a content of enhancing agent is 1 wt % to 20 wt %, and
a content of solvent is 45 wt % to 96 wt %.

7. The inhalable composition of claim 4, wherein the solvent comprises 98.5 wt % to 99.5 wt % water and 0.5 wt % and 1.5 wt % sodium chloride, based on a total weight of the solvent.

8. The inhalable composition of claim 6, wherein, based on a total weight of the inhalable composition, the enhancing agent comprises 1 wt % to 10 wt % glucose and 1 wt % to 10 wt % magnesium.

9. A device for supplying an inhalable composition, the device comprising:
a storage container storing an inhalable composition comprising caffeine and an enhancing agent comprising one or more selected from glucose and magnesium; and
an inhaler coupled to the storage container.

10. The device of claim 9, wherein the inhaler is a metered-dose inhaler, a dry powder inhaler, or a nebulizer.

11. The device of claim 9, wherein the inhalable composition further comprises vitamin B3 and at least one solvent selected from a group comprising water, alcohol, propylene glycol, and vegetable glycerin, and
the inhaler comprises an atomizer configured to atomize the inhalable composition.

12. The device of claim 11, wherein the atomizer is configured to atomize the inhalable composition using a jet method, an ultrasonic method, a vibration method, or a heating method.
